(19) United States
(12) Patent Application Publication
(10) Pub. No.: US 2022/0233226 A1
(43) Pub. Date: Jul. 28, 2022

(54) Distal Perfusion Cooling Mechanism

(71) Applicant: CryoTherapeutics SA, Awans (BE)

(72) Inventor: Hikmat Hojeibane, Princeton, NJ (US)

(21) Appl. No.: 17/581,097

(22) Filed: Jan. 21, 2022

Related U.S. Application Data
(60) Provisional application No. 63/140,800, filed on Jan. 23, 2021.

(51) Int. Cl. A61B 18/02

(52) U.S. Cl. CPC A61B 18/02; A61B 2018/0022

(57) ABSTRACT

A catheter includes a flexible heat transfer element, a cooling element encompassed by the flexible heat transfer element, a first tube couplable to a supply of coolant, and a second tube. The cooling element includes an elongate tubular wall defining an elongate cooling chamber therein having a first end that is coupled to a distal end of the second tube end and a second end that is closed. The distal end of the second tube is configured to receive a flow of coolant from the cooling element. A permeable distal portion of the first tube is positioned within the cooling chamber, the permeable distal portion including a plurality of holes in an outer surface of the tube such that when the coolant is supplied as a liquid at least some of the coolant passes through the plurality of holes and undergoes a phase change in the cooling chamber.

### CROSS-REFERENCE TO RELATED APPLICATIONS

The present application claims priority to U.S. Provisional Application Ser. No. 63/140,800, entitled “Distal Perfusion Cooling Mechanism” and filed on Jan. 23, 2021. The foregoing application is incorporated by reference herein in its entirety.

### BACKGROUND

From the late 1970s, catheters for cryotherapy have been used in the cardiovascular system starting from, for example, 1977 when it was used to surgically treat cardiac arrhythmias. Over the ensuing years it became widely recognised that cryotherapy was particularly advantageous for working in the heart. Its safety and efficacy was unsurpassed as surgeons were able to ablate delicate cardiac structures such as the A-V node, pulmonary veins and delicate peri-nodal atrial tissue without concern for thrombosis, perforation or other adverse events.

A catheter for the treatment of plaque stabilisation by cryotherapy is described in WO2015/067414A1 (referred to hereinafter as WO' 414). A balloon is inflated around a catheter shaft and subsequently cooled. A co-axially arranged cooling element is used to achieve this, wherein a liquid coolant is conveyed from an inner supply lumen into a larger conduit. When exiting the supply lumen, the coolant undergoes a phase change due to the pressure drop which occurs, causing it to evaporate and reduce in temperature. The cold gas is then removed using a return lumen which surrounds the supply lumen in a co-axial manner.

Accurate temperature control of such catheters is vital for ensuring patient safety. In practice, balloon-based cryotherapy catheters may have a non-uniform temperature distribution over the surface of the balloon. This can make it difficult to achieve accurate temperature control, as some areas of the balloon's surface are at a different temperature to others, and the temperature difference between areas is potentially unknown depending on the arrangement of temperature probes within the balloon. There is a need for catheters with improved temperature control.

### SUMMARY OF THE INVENTION

According to a first aspect of the invention, there is provided a catheter comprising a flexible heat transfer element provided on an outer surface of the catheter; a conduit arranged to supply an inflation fluid for inflating the flexible heat transfer element so as to form an inflated balloon; a cooling element encompassed by the flexible heat transfer element; a first tube having a proximal end that is couplable to a supply of coolant; and, a second tube, wherein the cooling element comprises an elongate tubular wall defining an elongate cooling chamber therein having a first end that is coupled to a distal end of the second tube end and a second end that is closed; wherein the distal end of the second tube is configured to receive a flow of coolant from the cooling element such that the second tube provides a flow path of the coolant from the distal end of the second tube to a proximal end of the second tube; and, wherein a permeable distal portion of the first tube is positioned within the elongate cooling chamber, the permeable distal portion comprising a plurality of holes in an outer surface of the first tube such that when the coolant is supplied via the proximal end of the first tube as a liquid at least some of the coolant passes through the plurality of holes and undergoes a phase change in the elongate cooling chamber.

Existing catheter designs generally deliver coolant through a supply lumen that has a single outlet at its distal tip. This means that the coolant is initially delivered to a very localised point inside the cooling element (and therefore the balloon), which in turn results in uneven cooling of the balloon due to the heterogeneous distribution of coolant within the cooling element. Due to the thermal resistance of the inflation fluid which is inside the balloon when the balloon is inflated, areas of the surface of the balloon which are further from the tip of the cooling element will not be cooled as quickly or effectively as those which are nearer the tip of the cooling tube. The surface of the balloon and any surrounding tissue will therefore cool unevenly.

The permeable distal portion of the present invention overcomes this problem by ensuring that the coolant is delivered more evenly within the elongate cooling element, which in turn results in more uniform cooling of the balloon. This therefore allows for improved control of the balloon's surface temperature by mitigating (potentially unknown) temperature variations on the surface of the balloon.

In addition, ensuring a more uniform distribution of coolant allows for longer balloons to be used. With existing catheters, the length of the balloon is limited by the rate at which heat can be transferred along the length of the cooling element and balloon. In contrast, the permeable distal portion of the present invention allows coolant to be delivered more evenly along the length of the balloon, thereby allowing longer balloons to be used.

The first tube may be referred to as a supply lumen or an injection lumen, and the second tube may be referred to as a return lumen or vacuum lumen. The conduit may be a shaft of the catheter (for example, the shaft of the catheter may encase the other lumens, and the conduit may be the space within the shaft between the other lumens), or it may alternatively be a separate inflation lumen.

Having the permeable distal portion on the first tube means the overall size of the cooling element does not need to be increased and also allows for small hole sizes to be used.

The permeable distal portion may be a perforated distal portion. In other words, the permeable distal portion may comprise perforations in the outer wall of the first tube. The holes may be laser drilled into the first tube, for example.

In preferred embodiments, a diameter of each of the plurality of holes is between 4 microns and 150 microns, and more preferably between 10 microns and 60 microns. In other preferred embodiments, the diameter of each of the plurality of holes is between 40 microns and 150 microns, preferably 60 microns to 150 microns, more preferably 80 microns to 150 microns. The holes may all be of the same size, or they may alternatively each have a different diameter.

Although the holes are preferably (substantially) circular in shape, they may be other shapes having a linear dimension of between (around) 4 microns and 150 microns. For example, the holes may have a cross-sectional area that is equivalent to that of a circle having a diameter between 4 microns and 150 microns. When the shapes are not circular, diameter should be understood to mean the distance between opposing edges of a perimeter of the hole.

The plurality of holes may be longitudinally and/or circumferentially spaced along the outer surface of the permeable distal portion of the first tube. In other words, the holes may be spaced along the length and/or around the circumference of the permeable distal portion of the first tube. Having the holes spaced longitudinally ensures uniform cooling along the length of the balloon, and having the holes spaced circumferentially ensures uniform cooling around the circumference of the balloon. The holes may be arranged with a regular spacing, or they may alternatively be irregularly or randomly/pseudo randomly spaced.

The permeability of the permeable distal portion may be graduated such that the permeability increases towards the distal end (the tip) of the permeable distal portion. For example, the holes may optionally increase in diameter towards the distal tip of the permeable distal portion. That is, the more distal holes may have a larger diameter than the more proximal holes. This helps to ensure that the coolant is dispersed evenly along the length of the permeable distal portion, instead of more coolant being dispersed at the more proximal holes. Additionally or alternatively, the density of the holes (that is, the number of holes per unit of surface area) may increase towards the distal end of the permeable distal portion to achieve a similar effect.

In some embodiments, the permeable distal portion may have a length of at least 1 mm and be at most as long as the balloon.

The permeable distal portion may be integral to the first tube (e.g., the permeable distal portion and the first tube may be formed of a single piece of material) or the permeable distal portion may be a separate or discrete component that is coupled directly to the first tube at the distal end of the first tube, e.g., the permeable distal portion may be glued, fused, or otherwise bonded to the first tube by any suitable means.

Preferably, the plurality of holes are arranged such that, in use, the inner surface of the balloon is cooled substantially uniformly by the cooling element around the circumference of the balloon and/or along the length of the balloon.

Preferably, the elongate cooling chamber has an interior cross-sectional area larger than an exterior cross-sectional area of the first tube and/or an exterior cross-sectional area of the permeable distal portion.

The elongate cooling chamber may additionally or alternatively have an interior cross-sectional area that is larger than an exterior cross-sectional area of the second tube.

In some embodiments, a guide wire lumen may extend through the balloon along a path that is external to both of the first and second tubes. In other words, the guide wire lumen may run adjacent to the first and second tubes.

In a preferred embodiment, the first tube and the second tube are coaxially arranged with each other such that, in a cross section of the co-axial arrangement of the first and the second tube, the first tube is enclosed by the second tube.

Preferably, the cooling element is arranged co-linearly with the distal end of the second tube.

Preferably, the cooling element is arranged co-linearly with the permeable distal portion.

Preferably, the elongate cooling element extends along a longitudinal axis in a direction parallel to a central axis of the balloon.

Optionally, the cooling element may be arranged inside the flexible heat transfer element such that, when viewed in the plane normal to the longitudinal axis of the cooling element, the cooling element is provided substantially within the centre of the balloon.

In some embodiments, the flexible heat transfer element provides the entire outer surface of the catheter for part of the catheter. That is, the flexible heat transfer element may form the outer surface of the catheter for a length of the catheter (e.g., for the length of the flexible heat transfer element).

The inflated flexible heat transfer element may occlude fluid flow between the walls of the vessel and the inflated flexible heat transfer element when in use in a vessel.

Alternatively, the flexible heat transfer element may provide part, but not all, of the outer surface of the catheter such that, when the catheter is inserted in a body and the flexible heat transfer element is inflated, blood can flow past the heat transfer element.

Preferably, the elongate cooling chamber is positioned within a central region of the balloon that is between a proximal end of the balloon and a distal end of the balloon.

Optionally, at least a part of the cooling element may be made of copper, silver, or gold.

In some embodiments, the flexible heat transfer element may be a perfusion balloon. Additionally or alternatively, the flexible heat transfer element may have a single walled outer membrane.

In use, the first tube and the second tube are preferably operated such that the pressure of the second tube is lower than the first tube. This pressure gradient means that the coolant preferentially returns along the second tube after being delivered by the first tube.

The coolant may be nitrous oxide, for example. The inflation fluid may optionally be a solution comprising one or more of water, sodium chloride, calcium chloride, ammonia, ethanol, propylene glycol, ethylene glycol, propanone and butanone.

Preferably, the catheter comprises a shaft for housing the first tube, the second tube, and the conduit of the catheter. Optionally, when the flexible heat transfer element is not inflated, the outer diameter of a part of the catheter that comprises the flexible heat transfer element is substantially the same as the outer diameter of the shaft of the catheter.

Preferably, the conduit is further configured to provide a return flow of the inflation fluid of the flexible heat transfer element. In some embodiments, the conduit comprises a third tube for providing a supply flow of the inflation fluid of the flexible heat transfer element. The conduit may also comprise a fourth tube for providing a return flow of the inflation fluid of the flexible heat transfer element.

Optionally, the catheter may comprise multiple cooling elements.

In some embodiments, the catheter may comprise means for heating the inflation fluid, or solidified inflation fluid, of the flexible heat transfer element. The means for heating may optionally be a resistor inside the flexible heat transfer element but outside the cooling element. The means for heating may additionally or alternatively electrodes configured to apply an electric current directly to the inflation fluid.

According to another aspect of the invention, there is provided a system for plaque stabilisation by cryotherapy, comprising a catheter according to the first aspect; an inflation device configured to supply inflation fluid to the conduit in order to inflate the flexible heat transfer element; a coolant source configured to supply coolant to the proximal end of the first tube; and, a vacuum pump configured to reduce the pressure in the second tube.

The system may further comprise means for monitoring the pressure inside the inflated flexible heat transfer element; and, means for determining if there has been any leakage of the inflation fluid from the catheter in dependence on the monitored pressure.

According to yet another aspect of the invention, there is provided a method for plaque stabilisation by cryotherapy, comprising supplying an inflation fluid to a catheter according to the first aspect to inflate the flexible heat transfer element of the catheter supplying a coolant to the proximal end of the first tube of the catheter; and, reducing the pressure in the second tube of the catheter.

The method may further comprise monitoring the pressure inside the inflated flexible heat transfer element; and, determining if there has been any leakage of the inflation fluid from the catheter in dependence on the monitored pressure.

### BRIEF DESCRIPTION OF THE DRAWINGS

Examples of the present invention will now be described in detail with reference to the accompanying drawings, in which:

FIG. 1 is a schematic illustration of a distal end of a catheter;

FIG. 2 is a schematic illustration showing a side view of a cooling element of the catheter;

FIG. 3 is a schematic illustration of a cross-sectional view of the cooling element;

FIG. 4 is a schematic illustration showing a cross-sectional view of the catheter;

FIG. 5 is an illustration of a system for operating the catheter; and,

FIG. 6 is a flowchart illustrating a method of operating the catheter.

### DETAILED DESCRIPTION

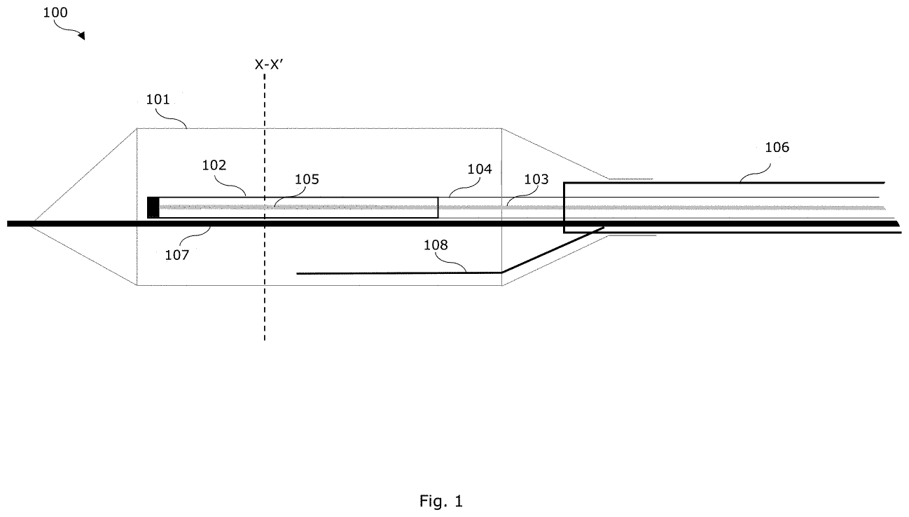

The distal end of an exemplary catheter 100 for cryotherapy is shown in FIG. 1. The catheter 100 is a balloon catheter and comprises a plurality of lumens supported inside a common shaft 106, except for in a balloon region where the lumens are instead provided within a flexible heat transfer element 101. The shaft 106 is preferably made of polyether block amide such as braided or unbraided PEBAX™, for example PEBAX 55D, and is preferably formed by extrusion or a heat reflow process.

The flexible heat transfer element 101, which may also be referred to as a flexible heat transfer membrane or balloon (when in the inflated configuration), is adhered to a distal tip of the shaft 106 on its proximal end and a guide wire lumen 107 on its distal end. The central region between these two ends is configured to be inflated into a substantially elongate, and substantially cylindrical balloon. Although the schematic illustration of FIG. 1 shows the flexible heat transfer element 101 extending from the shaft 106 at the proximal end of the balloon region at a sharp, constant angle up towards a cylindrical region (and back from this region towards the guide wire lumen 107 at the distal end), it should be understood that this profile is typically tapered, with the shape being primarily dictated by the elasticity and shape of the flexible heat transfer element 101, rather than any internal supporting members.

In FIG. 1, the flexible heat transfer element 101 is shown in an inflated configuration. The lumens of the catheter include a supply lumen 103 for providing a coolant to a cooling element 102 within the flexible heat transfer element 101, a return lumen 104 via which the coolant returns, and the guidewire lumen 107.

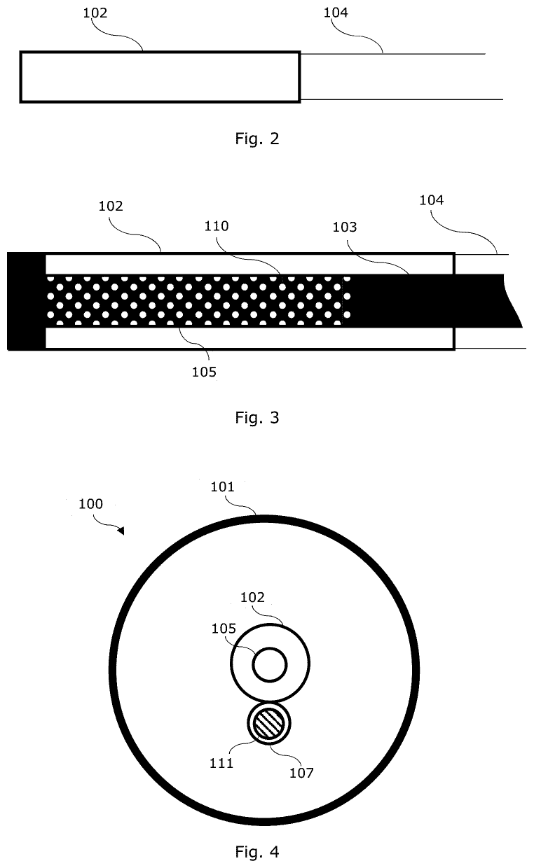

The cooling element extends 102 along the longitudinal axis of the balloon in a straight line until it terminates near the distal end of the balloon. The cooling element 102 is centrally arranged, and the flexible heat transfer element 101 is shaped such that, when the balloon is inflated, a substantially even cooling distribution is applied across the balloon. In particular, the cooling element 102 and the guide wire lumen 107 are arranged such that the outside of the balloon (i.e., the surface of the inflatable heat transfer element 101) is cooled quickly and uniformly. This is later discussed in more detail in connection with FIGS. 2 and 3.

The shaft 106, and in particular, the space between the lumens provided inside the shaft 106, acts as a supply and return path for providing a flow of inflation fluid to and from the balloon via the shaft 106. This enables space savings inside the shaft 106 since it is not necessary to provide a separate inflation and/or deflation tubes. Alternative embodiments are also envisaged in which the catheter has a dedicated inflation lumen for supplying an inflation fluid to/from the balloon. In some embodiments, the optional inflation lumen may supply inflation fluid to the balloon and inflation fluid may return via the space inside the shaft 106. Alternatively, the flow direction of the inflation fluid may be reversed such that the optional inflation lumen instead behaves as a deflation lumen whilst the shaft 106 is used to supply the inflation fluid.

The guide wire lumen 107 is configured to be threaded over a surgically implanted guide wire 111 (shown in FIG. 4) when in use for positioning the catheter 100 inside a patient. The guide wire lumen 107 extends from a guide wire entrance aperture at the distal tip of the catheter 100, to a guide wire exit aperture provided on the shaft 106 (as is standard for Rapid Exchange catheters). Alternatively, an ‘over the wire’ configuration may be used. The guide wire lumen 107 is a hollow tube and is preferably made of tri-layer or a similar material.

The catheter 100 also comprises a temperature probe 108 positioned within the flexible heat transfer element 101, which allows the temperature within the flexible heat transfer element 101 to be monitored. Temperature data collected by the temperature probe 108 can be utilised in controlling the supply of coolant. While the illustrated catheter 100 features a single temperature probe 108, additional temperature probes 108 may be used to allow temperatures at different locations within the flexible heat transfer element 101 to be monitored more accurately.

In use, the catheter 100 is inserted into a patient's body such that the balloon is positioned next to a region of tissue to be cooled in a vessel. For example, if the catheter is used for plaque stabilisation by cryotherapy, the balloon region may be positioned next to the plaque. Once the catheter 100 has been manipulated to the desired location within the patient's body, inflation fluid is pumped or otherwise driven through the shaft 106 (or optionally through an inflation lumen) to thereby fill and inflate the flexible heat transfer element 101 so as to form the inflated balloon.

The inflation fluid is preferably a liquid, and may be a solution that comprises sodium chloride, such as saline, with a sodium chloride concentration of about 0.9%, or a solution with a higher concentration of sodium chloride, preferably a 25% concentration of sodium chloride. The inflation fluid is preferably water based and may include various additives to lower the freezing point. Additives may include one or more of sodium chloride, calcium chloride, ammonia, ethanol, propylene glycol, ethylene glycol, propanone and butanone. Other additives may also be used, including contrast media. The inflation fluid is also preferably sterile. To ensure that the inflation fluid is sterile, the inflation fluid may be provided from a separate container, such as a pre-packed bag or syringe that is connected to the catheter.

Pressurised coolant is supplied to the cooling element 102 through the supply lumen 103 to thereby reduce the temperature of the cooling element 102. The coolant may be a liquid or a mixture of a liquid and a gaseous form of the coolant. The inflation fluid within the balloon is in thermal contact with the outer surface of the cooling element 102 and is thereby also cooled. During the procedure, coolant is preferably continually replenished by removing spent coolant through the return lumen 104, allowing a continuous supply of fresh coolant to be supplied in order to achieve and maintain the required cryogenic temperatures.

Turning now to FIG. 2, which shows a perspective view of the cooling element 102 of FIG. 1, the cooling element 102 is elongate and substantially cylindrical and comprises tubular walls. The walls of the cooling element 102 define an enclosed elongate cooling chamber within the cooling element 102. The cooling element 102 is coupled to the return lumen 104, which extends from the shaft 106, and is preferably unsupported within the balloon, but it may alternatively be attached to the guide wire lumen 107.

The supply lumen 103 (not visible in FIG. 2) is provided within the return lumen 104, and the supply lumen 103 and the return lumen 104 are each elongate and extend along respective axes that are substantially parallel to one another.

The supply lumen 103 and return lumen 104 are preferably made of reasonably strong materials so that they can withstand the pressure of a pressurised coolant. In some embodiments these lumens also have a degree of flexibility so that the catheter 101 can deform to match the profile of an artery or other blood vessel.

For example, the supply lumen 103 and return lumen 104 may be made of nylon, tri-layered tubing, polyimide, PEBAX™, such as PEBAX 55D. The supply lumen 103 and return lumen 104 may also be metal or polymer braided to add extra strength and flexible properties.

Furthermore, the cooling element 102 itself may be made entirely, or in part, of copper so that the cooling element has good thermal conductivity properties. Alternatively, the entirety of the cooling element 102, supply lumen 103 and return lumen 104 may be made from polyimide, enabling the walls to be made extremely thin. Using the same material for all of these components also improves the ease of manufacture.

Turning now to FIG. 3, which shows a close-up cross section view of the cooling element 102, the distal tip of the return lumen 104 may be closed by virtue of being coupled to the cooling element 102, whereas a permeable distal portion 105 at the tip of the supply lumen 103 has one or more holes or openings 110 in its outer wall such that coolant can flow from the supply lumen 103 in a first direction into the cooling element 102, and then flow in a second direction, opposite to the first direction, back along the return lumen 104.

The holes 110, which preferably each have a diameter of between 4 microns and 150 microns, may be formed by laser drilling or similar processes capable of forming micrometre-scale openings. Alternatively, the permeable distal portion 105 of the supply lumen 103 may be formed of a permeable membrane having holes or openings 110 with a diameter of between 4 and 150 microns.

The holes 110 in FIG. 3 are not shown to scale. In addition, although the illustrated holes 110 are shown as being substantially circular in cross section, the holes 110 could alternatively be other shapes.

Although the illustrated holes 110 all have a similar size, alternatives are envisaged in which the holes are of different sizes. For example, the holes 110 on the right side of FIG. 3 (i.e., the more proximal holes 110) may have a smaller diameter than the holes 110 on the left side of FIG. 3 (i.e., the more distal holes 110 closer to the tip of the catheter 100). Such an arrangement helps to ensure uniform dispersion of coolant along the length of the permeable distal portion 105, instead of having more coolant dispersed through the proximal holes 110. Additionally or alternatively, the number (density) of holes 110 may be higher towards the distal end.

In use, the coolant is supplied at cryogenic temperatures through the supply lumen 103. When the coolant reaches the permeable distal portion 105, it is forced through the holes 110 in the outer wall of the permeable distal portion 105. The cross-sectional area of the holes 110 is such that when the liquid coolant flows through the holes 110 the pressure drop caused by the restriction of the holes 110 causes the pressure of the liquid coolant to fall below its vapour pressure at the temperature of its surroundings at that point. This causes the liquid coolant to expand and preferably vaporise as it is forced through the holes 110, thereby leading to further cooling of the coolant.

The coolant, which may be nitrous oxide (N₂O), preferably enters the permeable distal portion 105 with substantially all of the coolant being in the liquid phase. The restriction caused by the holes 110 at the end of the supply lumen 103 ensures that there is little pressure drop within the supply lumen 103 and so most, or all, of the pressurised liquid coolant remains in the liquid phase in the supply lumen 103. The coolant may optionally exit the permeable distal portion 105 with some of the N₂O being in the liquid phase and some of the N₂O being in the gas phase.

The pressure within the return lumen 104, and thereby the cooling element 102, is preferably reduced by a vacuum pump. The vacuum pump, described in more detail later, operates at a proximal end of the return lumen 104. The reduction of pressure both increases the cooling effect due to expansion and phase change of the coolant and ensures that the coolant in the cooling element 102 flows into the return lumen 104.

Having the holes 110 distributed along the length of the permeable distal portion 105 ensures that the coolant is dispersed substantially uniformly/equally along the length of the cooling element 102 compared to designs having a single opening at the distal end of the supply lumen 103. This in turn ensures more uniform cooling along the length of the surrounding inflation fluid (and therefore the flexible heat transfer element 101). Likewise, having the holes 110 distributed circumferentially around the permeable distal portion 105 ensures that the coolant is dispersed substantially uniformly/equally circumferentially within the cooling element 102, thereby ensuring more uniform circumferential cooling of the surrounding inflation fluid and flexible heat transfer element 101.

The permeable distal portion 105 and supply lumen 103 may be integral components (i.e., the permeable distal portion 105 may be part of the supply lumen 103), or the permeable distal portion 105 may alternatively be a separate permeable component that is coupled to the supply lumen 103 at the distal end of the supply lumen.

Looking now to FIG. 4, which shows a cross-sectional view of the catheter 100 taken through the plane X-X′ of FIG. 1, the permeable distal portion 105 is arranged substantially centrally within the cooling element 102. The cooling element 102 is likewise positioned towards the centre of the flexible heat transfer element 101 in the illustrated example.

The guidewire lumen 107 is also positioned towards the centre of the flexible heat transfer element 101 and is adjacent to, and radially offset from the cooling element 107.

The central axis of the balloon will typically extend through the elongate cooling element 102, in the direction of the longitudinal axis of the cooling element 102. In embodiments where the balloon is elongate, for example, substantially cylindrical, the central axis of the balloon may also be referred to as the major axis of the balloon.

The longitudinal axis of the cooling element 102 is generally not exactly the same as the central axis of the balloon. It is advantageous to arrange the cooling element so that the thermal resistance between the cooling element 102 and the surface of the flexible heat transfer element 101 is uniform in all radial directions (i.e., all directions perpendicular to the longitudinal axis of the cooling element). To compensate for the presence of the guide wire lumen 107 (and any other lumens that may be provided inside the balloon), this may mean that the cooling element 102 is not exactly centred inside the balloon. The cooling element is therefore only ‘substantially central’ within the balloon. In some embodiments the longitudinal axis of the cooling element 102 may be distally offset from the central axis of the balloon by anything from 0 to 33% of the radius of the balloon, more typically 0 to 20%. For example, for a cylindrical 3 mm diameter balloon having a 1 mm diameter cooling element 102, the centre of the cooling element 102 may be up to 0.5 mm offset from the centre of the balloon, as viewed in a plane normal to the longitudinal axis of the cooling element 102. The exact position of the cooling element 102 within the balloon may vary slightly during use as the cooling element 102 may be free to vibrate inside the balloon.

The following dimensions may be desirable for certain applications that may include the treatment of plaque stabilization and atrial fibrillation on a human or animal:

Cooling element 102:
Outer diameter=0.35 to 1.20 mm
Outer wall thickness=0.019 to 0.080 mm
Length=15 to 30 mm

Return lumen 104:
Outer diameter=0.35 to 1.10 mm
Outer wall thickness=0.019 to 0.080 mm
Length=1000 to 1750 mm

Supply lumen 103 and permeable distal portion 105:
Outer diameter=0.12 to 0.40 mm
Outer wall thickness=0.014 to 0.080 mm
Length=1000 to 1750 mm (supply lumen), 1 to 30 mm (permeable distal portion)

Guide wire lumen 107:
Outer diameter=0.40 to 1.00 mm
Inner diameter=0.35 to 0.95 mm
Length=200-400 mm

Shaft 106:
Diameter=1.35 to 3.30 mm

Embodiments also include other dimensions, in particular the dimensions as provided in WO'414 which are incorporated herein by reference, and the above dimensions may be scaled up or down so that the catheter can be used with vessels of any size.

The flexible heat transfer element 101 is typically 10 mm to 37 mm long and, when deflated, is preferably substantially flush with the outer surface of the shaft 106 so that the outer diameter of the catheter 100 is not increased by the deflated balloon. For example, the outer diameter of the catheter 100 may be substantially 4 Fr (i.e., 1.333 mm). When inflated, the outer diameter of the flexible heat transfer element 101 may be 2.5 mm to 4 mm. However, a larger flexible heat transfer element 101 may be required in the treatment of atrial fibrillation with a diameter of approximately 24 mm (e.g., +/−10%). The dimensions of the catheter components, such as the lumen, may be adjusted depending on the application and, in particular, the size which the balloon is configured to be inflated. For example, larger lumens may be desired in order to inflate and deflate a large balloon more quickly or apply an increased cooling effect.

The flexible heat transfer element 101 may be made of a variety of materials and is desirably compliant or semi-compliant to ensure a good fit with the target area for effective heat exchange and a more even temperature distribution around the tissue. The flexible heat transfer element 101 may also be non-compliant if this is appropriate for the desired application. The flexible heat transfer element can be made of a variety of materials such as silicone or polyurethane for compliant balloons and nylon or polyester for non-complaint balloons. Wall thickness will also vary depending on the properties to be achieved, although they may be in the range of 5 to 100 microns for example. The balloon may also have a substantially smooth exterior surface so that heat transfer is optimised from the tissue on the interior surface of the vessel. The balloon material and thickness may be optimised to minimize thermal losses through the balloon wall.

As disclosed in WO2017/194557A1, the flexible heat transfer element 101 may be formed such that it inflates anisotropically (i.e., asymmetrically) when viewed in the plane normal to the longitudinal axis of the cooling element. In other words, the balloon may radially expand unevenly from the guide wire lumen 107 onto which it is secured so as to ensure that the cooling element 102 is substantially central within the balloon.

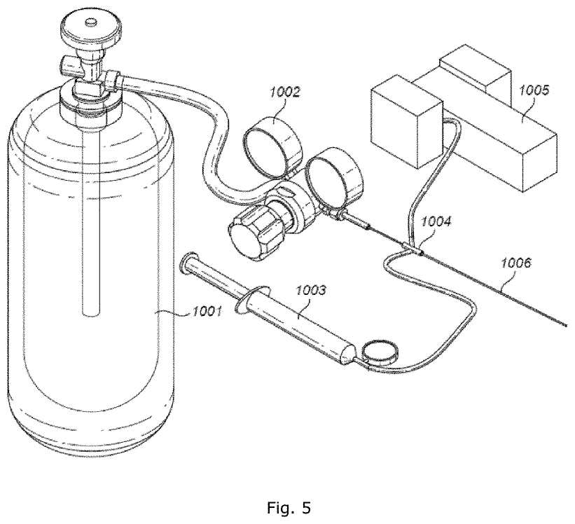

FIG. 5 is an illustration of an exemplary system for using the catheter 100 according to the examples described herein to cool a target part of a vessel. It will be understood that some of the specifically described components may not be essential to the operation of the system but are described for context only. Suitable, functionally similar, or equivalent components may be used interchangeably.

The system comprises:
Coolant cylinder 1001
Pressure regulator 1002
Tri-connector 1004
Vacuum pump 1005
Inflation device 1003
Catheter shaft 1006

Although not shown in FIG. 5, the system also comprises a catheter end according to any of the examples described herein. The coolant cylinder 1001 has a dip tube and spigot valve for controlling the supply of the coolant. A flexible high-pressure hose connects the coolant cylinder to the pressure regulator 1002. A supply tube from the pressure regulator connects to the tri-connector 1004. Also connected to the tri-connector is an inflation tube connected to inflation device 1003 and a return tube connected to the vacuum pump 1005. The tri-connector maintains the supply tube, the return tube and the inflation tube as separate from each other. The tri-connector also connects to the catheter shaft 1006 and thereby supports fluid and/or gas communication between the catheter and the coolant supply, vacuum pump, and inflation device.

The system may also include a heat exchanger, not shown in FIG. 5, to cool the liquid coolant before it enters the catheter. This will prevent boiling of the coolant as it enters the warm environment of the patient's body. Heat may be removed from the liquid coolant by using a refrigeration circuit or Peltier cooler.

The system may further comprise a computer, such that the system may be software controlled, the computer having one or more controls and/or a user interface such as a graphical user interface. The system may also further include assemblies for temperature and/or pressure monitoring based on signals received from one or more sensors.

The inflation device 1003 operates by causing an inflation fluid to flow into the catheter shaft 1006 when the plunger is pressed. The inflation device is also a deflation device since the inflation fluid flows back into the device from the catheter when the plunger is withdrawn. The inflation device may alternatively be an electric pump.

The vacuum pump 1005, which may be an electric vacuum pump, operates on the return lumens of the coolant. The vacuum pump 1005 advantageously lowers the pressure in the return lumen and/or cooling chamber of the cooling elements to thereby increase the amount of phase change of the coolant that occurs. The vacuum pump 1005 also ensures that the coolant in the supply lumens and cooling chambers (where provided) flows into the return lumen.

The system may also comprise a deflation device, separate from the inflation device 1003, that is in fluid communication with the catheter shaft 1006 through an additional separate connection to the tri-connector. The deflation device may be a vacuum pump, such as an electric vacuum pump. Variables that influence the operation of the catheter are the pressure of the inflated balloon and the temperature of the outer surface of the balloon. Both of these are controllable by how the system of FIG. 5 is operated. The pressure of the balloon is controllable by controlling the amount, and pressure of, the inflation fluid by inflation device 1003. The temperature of the outer surface of the balloon is dependent on both the temperature of the cooling element and how long the cooling element has been cooling the inflation fluid. The temperature of the cooling element is controllable by controlling the pressure and the amount of coolant that flows into the catheter. The length of time that the inflation fluid is cooled by the cooling element is easily controlled by when the system operator starts and stops the flow of the coolant into the catheter.

Preferably, the pressure of the balloon is maintained at lower than 5 ATM (507 kPa), typically between 2.5 ATM (253 kPa) to 4.5 ATM (456 kPa), but may be as low as 2 ATM (203 kPa) or 1 ATM (101 kPa). It may be desirable for the balloon pressure to be as low as possible for effective treatment in order to mitigate the risk of a reaction occurring in the blood vessel that leads to restenosis or blockage. A short-term response to the application of high-pressure cryotherapy is also often smooth muscle cell proliferation, which is potentially dangerous. The tissue interface temperature is preferably maintained within a desired range in order to remove heat from the plaque and vessel without significantly ablating the cells. It is noted that throughout the present document, all pressures given as gauge pressures, that is, above atmospheric pressure.

The temperature of the outer surface of the balloon is maintained within appropriate ranges for the given application. For example, for cryotherapy, the temperature is preferably maintained between +15° C. (288K) and −35° C. (238K) and more preferably between 0 to −30° C. (273K to 243K). For atrial fibrillation (and other applications where tissue ablation is required) the temperature may be much lower, for example between −50° C. to −90° C. (223K to 183K), although typically around −80° C. (193K).

The exact temperature will depend on the treatment application, according to standard practice. Depending on the type of balloon and the heat load, there may be a temperature difference of about 10° C. to 40° C. between inner and outer balloon temperature and this can be compensated for when controlling the system. Preferably, sensors such as the temperature probe 108 are provided within, on or near the catheter end, such as on or just inside the balloon, in order to monitor and thereby control the temperatures and pressures in a feedback control system.

For example, a thermocouple may be fixed to the guide wire lumen or coolant return tube to measure the temperature inside the balloon. One or more further thermocouples may be attached to the internal or external surface of the balloon in order to measure the balloon tissue interface temperature.

In addition, a pressure sensor may be placed inside the balloon to accurately monitor and thereby control the pressure within the balloon. The pressure sensor may be an open hydraulic tube with no flow, or may be positioned on the inflation circuit near the inflator, so that the fluid pressure inside the tube is measured outside the catheter. The pressure sensor may also be a piezoelectric transducer, fibre-optic transducer or other type of sensor. Pressure sensors and a flow meter may also be positioned in the coolant circuit, to measure the pressure and flow of the coolant. Both temperature and pressure signals can be used to control refrigerant flow such that balloon pressure and/or surface temperature remain within the desired ranges. The pressure transducer may also be used to detect any leaks within the catheter by sensing abnormal pressures. The temperature sensor(s) may also be used to detect vessel occlusion by the balloon.

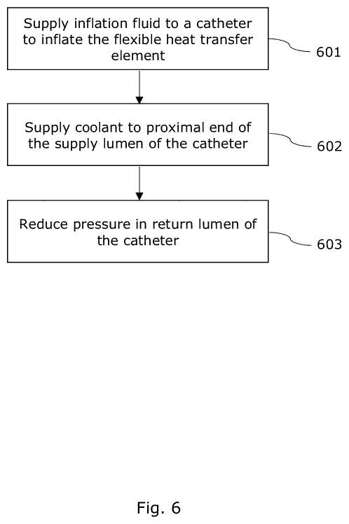

FIG. 6 illustrates an exemplary method for operating the catheter 100. At step 601, an inflation fluid is supplied to the catheter 100 via the shaft 106 to inflate the flexible heat transfer element 101. At step 602, the coolant is supplied to the proximal end of the supply lumen 103 of the catheter. At step 603, the pressure in the return lumen 104 of the catheter 101 is reduced. It should be understood that steps 602 and 603 could be performed simultaneously or in the reverse order.

Embodiments of the catheter 100 include a number of modifications and variations that can be made to the embodiments as described above. In particular, the dimensions provided are approximate and embodiments include catheter designs with different dimensions. Furthermore, the dimensions may also vary depending on the size of the human or animal that is being treated. Throughout the present document various features are described as lumens and tubes. These terms may be used interchangeably, and these features may also be referred to as conduits. In the above described embodiments, the cooling element is preferably substantially straight and co-linear with the abutting end of the shaft. The cooling element may be rigid and non-flexible. However, the cooling element is preferably flexible so that it can bend, as is appropriate if the balloon is positioned in a curved section of a blood vessel.

In the above-described operation of the system, operational temperatures and pressures are provided. However, embodiments are in no way limited to these operational temperatures and pressures. Moreover, the operational temperatures and pressures may be varied depending on the application. In particular, embodiments include the catheter, and the system supporting the catheter, being operated according to the disclosure in WO2012/140439A1, the entire contents of which are incorporated herein by reference.

Reference to the term distal should be understood to mean an end of a catheter, lumen, tube or other instrument that is inserted into a patient, whereas the term proximal should be understood to be the opposing end of the catheter, lumen, tube or other instrument. For example, the proximal end of a catheter may be connected to control apparatus.

1. A catheter comprising:
a flexible heat transfer element provided on an outer surface of the catheter;
a conduit arranged to supply an inflation fluid for inflating the flexible heat transfer element so as to form an inflated balloon;
a cooling element encompassed by the flexible heat transfer element;
a first tube having a proximal end that is couplable to a supply of coolant; and,
a second tube,
wherein the cooling element comprises an elongate tubular wall defining an elongate cooling chamber therein having a first end that is coupled to a distal end of the second tube end and a second end that is closed;
wherein the distal end of the second tube is configured to receive a flow of coolant from the cooling element such that the second tube provides a flow path of the coolant from the distal end of the second tube to a proximal end of the second tube; and,
wherein a permeable distal portion of the first tube is positioned within the elongate cooling chamber, the permeable distal portion comprising a plurality of holes in an outer surface of the first tube such that when the coolant is supplied via the proximal end of the first tube as a liquid at least some of the coolant passes through the plurality of holes and undergoes a phase change in the elongate cooling chamber.

2. The catheter of claim 1, wherein a diameter of each of the plurality of holes is between 4 microns and 150 microns, preferably between 10 microns and 60 microns.

3. The catheter of claim 1, wherein a diameter of each of the plurality of holes is between 40 microns and 150 microns, preferably 60 microns to 150 microns, more preferably 80 microns to 150 microns.

4. The catheter of claim 1, wherein the plurality of holes are longitudinally spaced along the outer surface of the permeable distal portion of the first tube and the holes of the plurality of holes at a distal end of the permeable distal portion have larger respective diameters than holes of the plurality of holes at a proximal end of the permeable distal portion.

5. The catheter of claim 1, wherein the plurality of holes are circumferentially spaced around the outer surface of the permeable distal portion of the first tube.

6. The catheter of claim 1, wherein a length of the permeable distal portion of the first tube is at least 1 mm.

7. The catheter of claim 1, wherein the permeable distal portion is integral to the first tube.

8. The catheter of claim 1, wherein the permeable distal portion is a discrete component that is coupled directly to the first tube at the distal end of the first tube.

9. The catheter of claim 1, wherein the elongate cooling chamber has an interior cross-sectional area larger than an exterior cross-sectional area of the first tube.

10. The catheter of claim 1, wherein the elongate cooling chamber has an interior cross-sectional area larger than an exterior cross-sectional area of the second tube.

11. The catheter of claim 1, wherein a guide wire lumen extends through the balloon along a path that is external to both of the first and second tubes.

12. The catheter of claim 1, wherein the first tube and the second tube are coaxially arranged with each other such that, in a cross section of the co-axial arrangement of the first and the second tube, the first tube is enclosed by the second tube.

13. The catheter of claim 1, wherein the elongate cooling element extends along a longitudinal axis in a direction parallel to a central axis of the balloon.

14. The catheter claim 1, wherein the cooling element is arranged inside the flexible heat transfer element such that, when viewed in the plane normal to the longitudinal axis of the cooling element, the cooling element is provided substantially within the centre of the balloon.

15. The catheter of claim 1, wherein the elongate cooling chamber is positioned within a central region of the balloon that is between a proximal end of the balloon and a distal end of the balloon.

16. The catheter of claim 1, wherein the flexible heat transfer element is a perfusion balloon.

17. The catheter of claim 1, wherein the flexible heat transfer element has single walled outer membrane.

18. The catheter of claim 1, wherein, in use, the first tube and the second tube are operated such that the pressure of the second tube is lower than the first tube.

19. The catheter of claim 1, further comprising means for heating the inflation fluid, or solidified inflation fluid, of the flexible heat transfer element.

20. A method for plaque stabilisation by cryotherapy, comprising:
supplying an inflation fluid to a catheter according to claim 1 to inflate the flexible heat transfer element of the catheter;
supplying a coolant to the proximal end of the first tube of the catheter; and,
reducing the pressure in the second tube of the catheter.
